(19) United States
(12) Patent Application Publication
(10) Pub. No.: US 2007/0178582 A1
(43) Pub. Date: Aug. 2, 2007

(54) Microfabricated cellular traps based on three-dimensional micro-scale flow geometries

(76) Inventor: Hur Koser, New Haven, CT (US)

Correspondence Address: Arthur G Schaier;Carmody & Torrance, 50 Leavenworth Street, P O Box 1110, Waterbury, CT, 06721-1110, US

(21) Appl. No.: 10/589,653

(22) PCT Filed: Feb. 16, 2005
(86) PCT No.: PCT/US05/05000

§ 371 Date: Aug. 16, 2006

Related U.S. Application Data
(60) Provisional application No. 60/545,615, filed on Feb. 17, 2004.

(51) Int. Cl. C12M 1/34

(52) U.S. Cl. 435/288.5; 435/297.5

(57) ABSTRACT

An improved microfluidic device and method of using the microfluidic device to measure natural motile response of a living moiety to a chemotactic agent. The invention comprises integrating microfluidic containment trenches within the microfluidic devices for cellular trapping, manipulation and real-time analysis of biological phenomena, including chemotaxis. The invention captures the design methodology for these traps, as well as the fabrication means and the associated external control mechanism that allows rapid, reversible and fully controlled changes in the chemical environment to which trapped cells are exposed.

### CROSS-REFERENCE TO RELATED APPLICATIONS

This application claims the benefit of U.S. Provisional Application No. 60/545,615, filed Feb. 17, 2004, the subject matter of which is herein incorporated by reference in its entirety.

### FIELD OF THE INVENTION

The invention is directed to an improved microfluidic device and a method of using the microfluidic device to measure natural motile response of a living moiety to a chemotactic agent.

### BACKGROUND OF THE INVENTION

Chemotaxis is a fundamental cellular process that describes the motile response of cells to the presence of a concentration gradient of a given chemical in solution. Of particular interest is the creation of efficient, highly controllable chemical gradient pattern generation chambers fabricated using soft lithography techniques, in order to quantify chemotaxis in virtually any cell. Until very recently, fundamental shortcomings in experimental apparatus allowed only for the determination of the presence or absence of a chemotactile response in a population of cells, while precluding its exact quantification on the level of an individual cell. Efficient, highly controllable chemical gradient pattern generation chambers fabricated using soft lithography techniques offer attractive alternatives to existing macro-scale devices. These devices utilize laminar flow and rapid diffusion that are characteristic in micro-scale flow channels to create spatially and temporally stable chemical concentration patterns.

Efficient, highly controllable chemical gradient pattern generation chambers fabricated using soft lithography techniques offer an attractive alternative to existing macro-scale devices. While such microfluidic devices have recently been demonstrated, their entire potential as efficient drug discovery platforms has not been exploited yet. This is partly because the chemical gradient within the microfluidic device can only be maintained with continuous flow, and most cells require special substrate surface chemistry to maintain their positions within the field of view of a microscope objective. The process of chemically modifying the substrate surface for a particular cell type is complex, may be costly and difficult. Even then, the shear force of the flow over the membrane of the cell is disruptive.

Prior art research efforts, such as those described by Jeon et al., “Neutrophil Chemotaxis in Linear and Complex Gradients of Interleukin-8 Formed in a Microfabricated Device,” Nature Biotechnology, Volume 20, Number 8, pp. 826-830, August 2002, the subject matter of which is herein incorporated by reference in its entirety, have studied chemotaxis on rabbit neutrophils stuck to a glass substrate. However, not all cell types, move in an amoeba-like fashion like neutrophils.

For instance, bacteria move via a set of propelling organs, such as a flagella or cilia. A bacterium responds to chemical gradients of chemo-attractants (or chemo-repellents) by performing a biased random walk up (or down) of the gradient, with short periods of straight swimming interrupted by tumbling that reorients the cell preferentially towards the gradient. For that reason, surface chemistry modification alone does not result in microfluidic chemotaxis chambers compatible with bacteria. The flow regimes that are practically achievable for sustaining chemotactic gradients in fluidic channels are on the order of a micron per second or faster (approximately 1 mm/s is the norm); within the resolutions needed to observe bacteria under the microscope, the cells simply flow out of the field of view in seconds. What is needed to observe and quantify chemotaxis in bacteria effectively is a means of exposing them to the concentration gradient of the solute of interest while keeping them long enough under the field of view.

Furthermore, existing methods of cell trapping and manipulation within microfluidic devices, such as dielectrophoresis, expose the cells to spatially non-uniform external forces that prove disruptive in efforts to measure the natural motile response of the cells. The ideal chamber that is suited to all types of cells, including bacteria, would provide some means of essentially freezing the chemotactic gradient locally, so that the cells are free to move and respond naturally to the chemotactic agent.

Over the recent decades, technology originally developed to bulk produce semiconductor devices such as microprocessors has been successfully applied in the manufacture of miniature mechanical sensors and actuators that are known today as micro-electromechanical systems (MEMS). Some MEMS devices, such as accelerometer chips in car airbag inflation systems, miniature pressure sensors, gyroscopes, and ultra bright display chips have already revolutionized their respective markets. Lately, MEMS fabrication techniques have been applied towards the creation of fluidic devices with micron-scale features. These microfluidic devices offer the potential to integrate many different aspects of a chemical or bioengineering laboratory onto a single chip (thereby functioning as a “lab-on-a-chip”) that is able to handle miniscule amounts of sample and produce highly accurate and speedy results. Thanks to a stamping and molding process involving poly-dimethylsiloxane (PDMS), the prototyping process for microfluidic devices is rather simple, rapid and cheap.

Microfluidic devices made out of PDMS are robust, easy-to-handle, and most importantly, disposable. Currently, they are a popular medium of choice for lab-on-a-chip research. High levels of integration, including hundreds of control valves, separation chambers have already been demonstrated in PDMS devices.

The inventors of the present invention have developed a means of integrating microfluidic containment trenches within microfluidic devices for cellular trapping, manipulation and real-time analysis of biological phenomena, including chemotaxis. The invention captures the design methodology for these traps, as well as the fabrication means and the associated external control mechanism that allows rapid, reversible and fully controlled changes in the chemical environment to which trapped cells are exposed.

### BRIEF DESCRIPTION OF THE FIGURES

FIG. 1 depicts a view of a microfluidic device of the invention that is formed from a stack of i) glass; ii) SU-8 (a photoresist available from MicroChem); and iii) PDMS (the resulting device is transparent and compatible with optical microscopy). The main flow channel is used to create the desired chemical environment, while the side inlets introduce solutions containing individual cells into specialized containment trenches.

FIG. 2 provides a schematic of a sample linear gradient pattern generator. At each level, the length of the branches is chosen long enough to ensure complete mixing.

FIG. 3 presents a cut-away view of one embodiment of the microfluidic device of the invention depicting a cell containment trench. For illustration purposes, vertical dimensions and the size of the diffusion holes are exaggerated. Thanks to rapid diffusion across the smaller two dimensions of the trench, the main flow continuously replenishes and controls the chemical environment under the “'jail bars.”

FIG. 4 depicts a numerical simulation result overlaid over a schematic side view of a cell confinement trench underneath the main flow channel. The average flow rate within the trench is just a few microns per second.

FIG. 5 demonstrates that using smaller, multiple diffusion holes across the cross-section reduces the average flow rate within the trench to under 1 μm/s.

FIG. 6 depicts a typical fabrication process for both the trench geometry and the PDMS main flow channel components.

### SUMMARY OF THE INVENTION

The present invention is directed to a microfluidic device for measuring natural motile response of a living moiety to a chemotactic agent, comprising:

a) a flow channel for transporting the chemotactic agent through the microfluidic device;

b) at least one microfluidic trench arranged beneath and substantially perpendicular to the flow channel, wherein the living moiety is introducible into the at least one microfluidic trench; and

c) means for measuring the response of the living moiety to the chemotactic agent;

wherein the chemotactic agent is introducible into the at least one microfluidic trench to expose the living moiety to the chemotactic agent, and wherein the flow of the chemotactic agent over the at least one microfluidic trench creates a hydrodynamic stagnation of flow within the at least one microfluidic trench.

In a preferred embodiment, the chemotactic agent is introducible into the at least one microfluidic trench by diffusion.

In one embodiment, the microfluidic device comprises one or more means for trapping the living moiety within the at least one microfluidic trench.

In another preferred embodiment, the microfluidic device comprises means for creating a controlled concentration gradient of the chemotactic agent prior to introducing the chemotactic agent into the flow channel.

The invention is also directed to a method of using a microfluidic device to measure natural motile response of a living moiety to a chemotactic agent, the method comprising the steps of:

a) transporting the chemotactic agent through the microfluidic device in a flow channel;

b) introducing at least one living moiety into at least one microfluidic trench arranged beneath and substantially perpendicular to the flow channel; and

c) measuring the response of the at least one living moiety to the chemotactic agent;

wherein the chemotactic agent is introduced into the at least one microfluidic trench to expose the at least one living moiety to the chemotactic agent, and wherein the flow of the chemotactic agent over the at least one microfluidic trench creates a hydrodynamic stagnation of flow within the at least one microfluidic channel.

In another embodiment, the invention is directed to a method of fabricating a microfluidic device comprising the steps of:

a) providing a glass slide;

b) fabricating a layer comprising at least one microfluidic trench on the glass slide, wherein an opening of the at least one microfluidic trench is opposite the surface of the glass slide;

c) providing a roof structure layer on the layer comprising the at least one microfluidic trench, wherein said roof structure comprises a plurality of openings; and

d) providing a top layer comprising a flow channel, wherein said flow channel is open to the roof structure;

wherein a fluid introduced into the flow channel is introducible into the at least one microfluidic trench through the roof structure layer.

### DETAILED DESCRIPTION OF THE INVENTION

The present invention is rather simple in its basics and yet quite fundamental in developing the ability to manipulate cells chemically in microfluidic environments. The cellular trapping mechanism of the microfluidic device of the invention, its fabrication process and the associated external hardware that make up the microfluidic device provide a very useful invention.

The inventors of the instant application have developed a novel microfluidic chemotaxis chamber that accommodates fast, efficient, repeatable, parallel measurements on virtually any living moiety, especially motile cells.

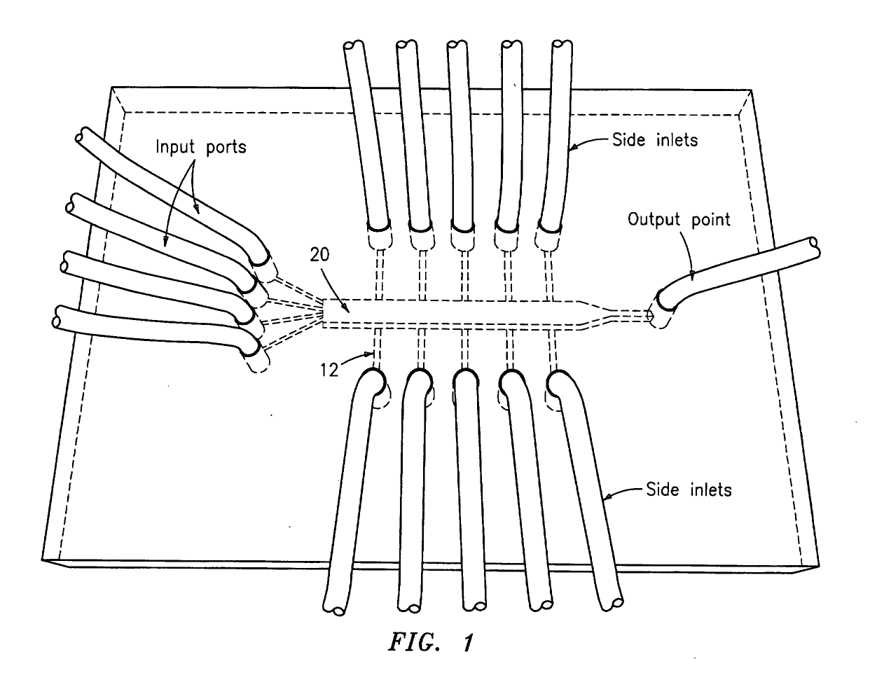

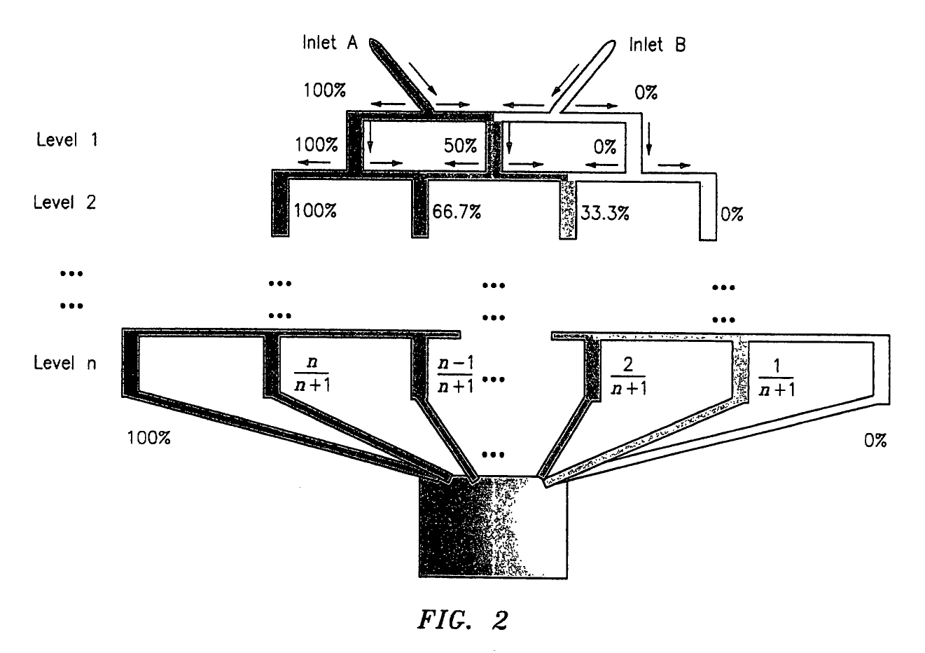

The microfluidic device itself is composed of three distinct, coupled functional regions. The first compartment is positioned just after the fluidic inputs to the device, and is involved in creating the chemical gradient. This region's geometry may be as simple as multiple input channels merging to form the main channel (essentially, what is depicted in FIG. 1), or as complicated as the method described by Jeon et al., where a small number of input ports are used to create sophisticated chemical concentration gradients, as illustrated in FIG. 2. The common underlying mechanism involves laminar flow and very rapid diffusion across micro-scale dimensions, in order to achieve controlled mixing of the contents of the input ports.

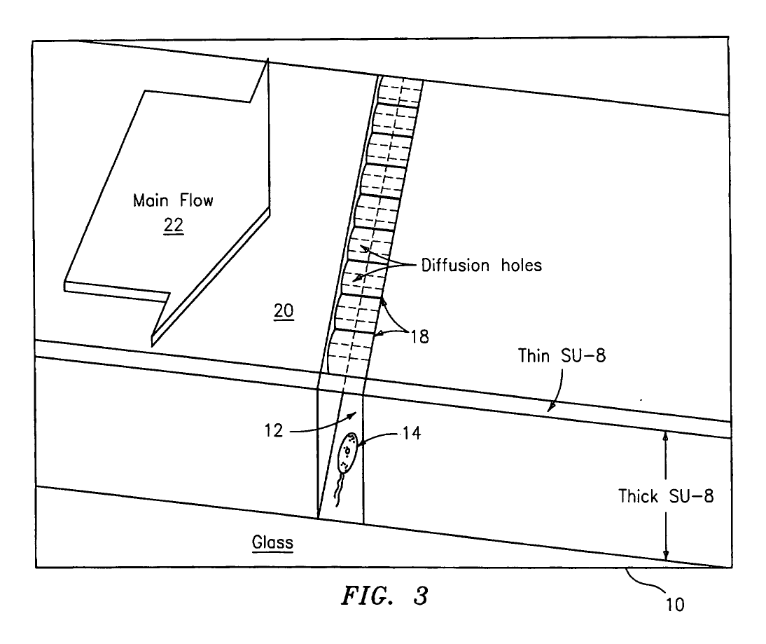

The main flow channel that follows is wide enough to ensure that diffusion across its width is relatively slow and, given the flow speed, the concentration profile achieved at the input section is essentially maintained throughout its length. In a preferred embodiment the main flow channel has a width of about 50 microns to about 2 millimeters and a length of about 500 microns to a few centimeters. The final set of compartments is positioned underneath the main flow chamber, lying as perpendicular trenches across its width; these are the microfluidic containment trenches that are designed to sustain the individual motile cells as depicted in FIG. 3. The containment trenches typically have a width and height range between about 5 and 50 microns. As is readily apparent, the length of the microfluidic trenches is approximately equal to the width of the main flow channel.

The basic purpose of the trenches underneath the main flow channel, as shown in FIG. 3, is to provide a mechanism to confine cells, which are initially introduced through the side inlets. Since the Reynolds' numbers associated with the flow are very small (even for the maximum practically achievable flow rates), the main flow stays laminar over the trench geometry. However, extensive simulations of Navier-Stokes equations have shown that appropriately chosen trench dimensions (the width and the height) result in the formation of a local eddy within the confinement trench. It has been confirmed that these local eddies tend to form virtually at any operating flow rate given suitable trench dimensions. What is more, the average flow rate within the trench is then orders of magnitude slower than the flow in the main channel.

Flow within the microfluidic trenches is also generally orders of magnitude slower than (essentially stagnant compared to) the flow in the main channel above them and thus hydrodynamic stagnation of flow within the microfluidic trench occurs. The dimensions are easily customizable to make sure that the cells inside the microfluidic trenches can move faster than the flow around them. As long as the depth of the microfluidic trenches are much smaller than the width of the main flow channel, the concentration gradient in the flow channel diffuses down and is established along the length of the microfluidic trenches as well. Furthermore, the inventors have determined that it is highly desirable that the values for width and height of the microfluidic trenches be much smaller than the value for the length of the microfluidic trenches. Therefore, the cells within the microfluidic trenches may be exposed to the desired chemotactic gradient, without appreciable disruptive shear forces over their membranes.

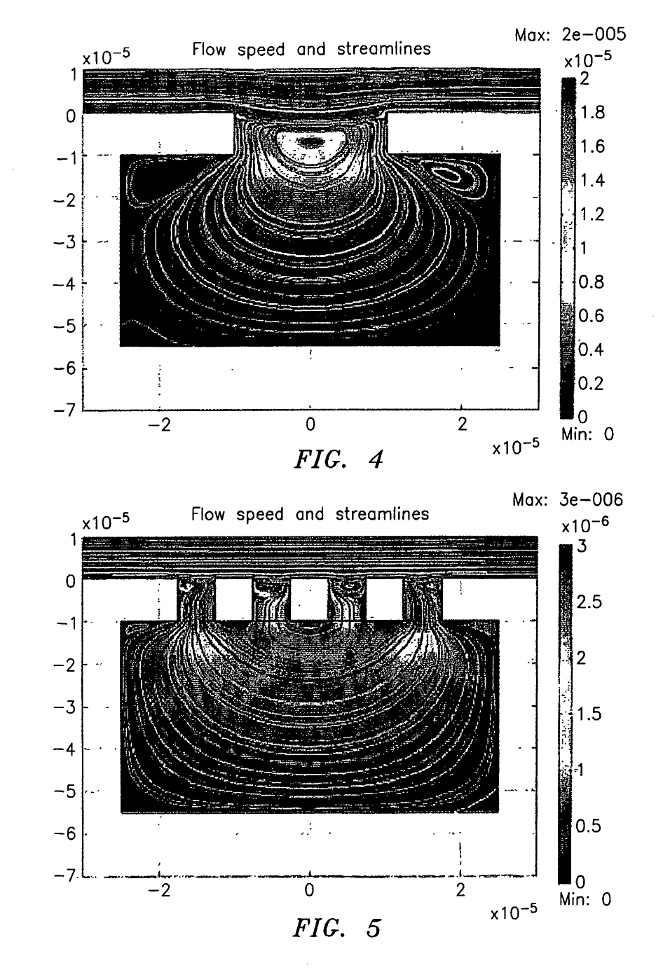

FIG. 4 illustrates the flow profile just over and inside a 50 μm×50 μm trench, covered on top by a 20 μm opening. The average flow rate within the main channel is 1 mm/s, whereas the average flow speed inside the trench is just a few microns per second. Introduction of multiple, smaller openings (i.e., a roof structure) on top across a given trench cross-section reduces the flow rate inside the trench to less than 1 μm/s as seen in FIG. 5. This geometry then allows experiments in which cells can swim virtually freely along the length of the trench without experiencing any appreciable drag or shear forces from the flow. Using standard MEMS fabrication techniques, the holes of the roof structure can easily be chosen to be smaller than the dimensions of the trapped cells, so as to guarantee that the cells will not accidentally swim out of the confinement trenches.

The other advantage of this geometry is that the gradient pattern established in the main flow is preserved spatially within the trench. This is because both the nutrients and the chemotactic concentration gradient within the main flow above continuously diffuse through the diffusion holes. As long as the depth of the trenches are much smaller than the width of the main flow channel this diffusion is complete. Therefore, the cells within the confinement trenches can be exposed to the desired chemotactic gradient.

This simple approach is effective for observing chemotaxis on relatively large, slow moving cells, such as lymphocytes. For smaller, faster moving organisms, the trenches may be capped on top by a roof structure. For example, the roof structure may comprise a thin layer of SU-8, with periodic thin slots (“jail bars”) that allow the concentration gradient to diffuse through, while preventing the trapped cells from escaping.

One of the main features of this technology is the incorporation of cell trapping trenches that use the formation of local eddy-current flows to create calm, cell sustaining regions just underneath a relatively fast moving flow region. The main advantage of this approach is that the fast moving flow region can support a stable, controlled chemical gradient just over the trench, while exerting virtually no disturbing forces on the trapped cells. The chemical gradient and nutrients present in the main flow channel diffuse down into the trenches very effectively when the dimensions of the trench are small compared to those of the main flow channel. This approach eliminates any need for expensive and time-consuming surface chemistry adjustments that would otherwise be required for each different species of cells. In this way, multiple trenches can be formed on the same substrate, and multiple cell species can be supported simultaneously.

Although chemotaxis gradient pattern generators have been demonstrated previously, effective, completely non-intrusive cell trapping mechanisms to integrate with them have not.

The inventor of the present invention has designed, fabricated and tested a microfluidic chemotaxis chamber that accommodates fast, efficient, repeatable, parallel measurements on virtually any motile cell. The resulting chambers house the cellular signaling and programming nanotools that are described above, as well as the integrated electrical signaling pathways that may be necessary. Hence, the microfluidic chambers will allow the direct manipulation of individual cells through an ability to precisely control the chemical environment that the cells occupy.

The general architecture of the proposed microfluidic devices is illustrated in FIGS. 1 and 3. The geometric details and dimensions of the micro-channels are variable, depending on the particular cell size and type, as well as the specific chemical environment that is desired. A fluid dispensing system, including miniature electronically-controlled valves, precede the input ports that carry the desired chemical solutions to the main flow channel. Such a system can be built in-house using a simple pressure-based driving mechanism and miniature solenoid valves. There are also commercially available and highly accurate micro-dispensing systems such as the Multiplex® system, available from IVEK Corporation. Such a system allows the user to control flow velocities accurately and to change flow compositions rapidly. A separate system controls the side inlets through which the bacteria or other cells of interest are introduced into the microfluidic “trapping” trenches just underneath the main channel.

As depicted in FIG. 3, the present invention is directed to a microfluidic device (10) for measuring natural motile response of a living moiety (14) to a chemotactic agent (22). The device comprises a flow channel (20) for transporting the chemotactic agent (22) through the microfluidic device (10), and at least one microfluidic trench (12) arranged beneath and substantially perpendicular to the flow channel (20), wherein the living moiety (14) is introducible into the at least one microfluidic trench (12), and means for measuring the response of the living moiety to the chemotactic agent. The means for measuring the response of the living moiety to the chemotactic agent are not shown but may be as simple as observing and recording the reaction of the living moiety to the chemotactic agent through a microscope. The chemotactic agent (22) is introducible into the at least one microfluidic trench (12) to expose the living moiety (14) to the chemotactic agent (22). The flow of the chemotactic agent (22) over the at least one microfluidic trench (12) creates a hydrodynamic stagnation of flow within the at least one microfluidic trench (12). In a preferred embodiment, the chemotactic agent (22) is introducible into the at least one microfluidic trench (12) by diffusion.

The microfluidic device of the invention also comprises means for creating a controlled concentration gradient of the chemotactic agent prior to introducing the chemotactic agent into the flow channel, as discussed in more detail above.

Preferably, the at least one microfluidic trench has a length dimension that is much larger than its dimensions for width and depth. Typically, the living moiety is selected from the group consisting of bacteria and cell species and the at least one microfluidic trench allows for full motility of the living moiety within the at least one microfluidic trench.

The microfluidic device may also comprise means for trapping the living moiety (14) in the at least one microfluidic trench (12). This may be accomplished in a variety of ways. For example, in order to keep the living moiety in the microfluidic trench once the living moiety have been inserted into the at least one microfluidic trench (12) through an inlet, each end of the microfluidic trench nay be sealed by means of a mechanical microvalve or an air valve (not shown). Other suitable means would also be known to one skilled in the art. In addition, in order to keep the living moiety from escaping out of the top of the at least one microfluidic trench (12), a roof structure (18) may be placed over the at least one microfluidic trench (12). The roof structure (18) generally comprises a patterned substrate surface that traps the living moiety within the at least one microfluidic trench while allowing the chemotactic agent to be introduced into the at least one microfluidic trench. In a preferred embodiment, the roof structure comprises a semi-permeable membrane made of polycarbonate or polyethylene.

In one embodiment of the invention, multiple microfluidic devices may be used and the microfluidic device communicates with at least one additional microfluidic device as part of a system of devices through the control of microvalves and micropumps.

In another embodiment, the microfluidic device may comprise means of applying a temporally varying electric field to group, separate or select specific living moieties within the at least one microfluidic trench. For example, the electrical field may be applied to move the group of living moiety to one side of the trench and then measure which of the living moiety in the trench reacts most quickly to the chemotactic agent. The living moiety that reacts most quickly may then be removed and replicated.

The present invention is also directed to a method of using a microfluidic device to measure natural motile response of a living moiety to a chemotactic agent. The method typically comprises the steps of:

a) transporting the chemotactic agent through the microfluidic device in a flow channel;

b) introducing at least one living moiety into at least one microfluidic trench arranged beneath and substantially perpendicular to the flow channel; and

c) measuring the response of the at least one living moiety to the chemotactic agent;

wherein the chemotactic agent is introduced into the at least one microfluidic trench to expose the at least one living moiety to the chemotactic agent, and wherein the flow of the chemotactic agent over the at least one microfluidic trench creates a hydrodynamic stagnation of flow within the at least one microfluidic channel.

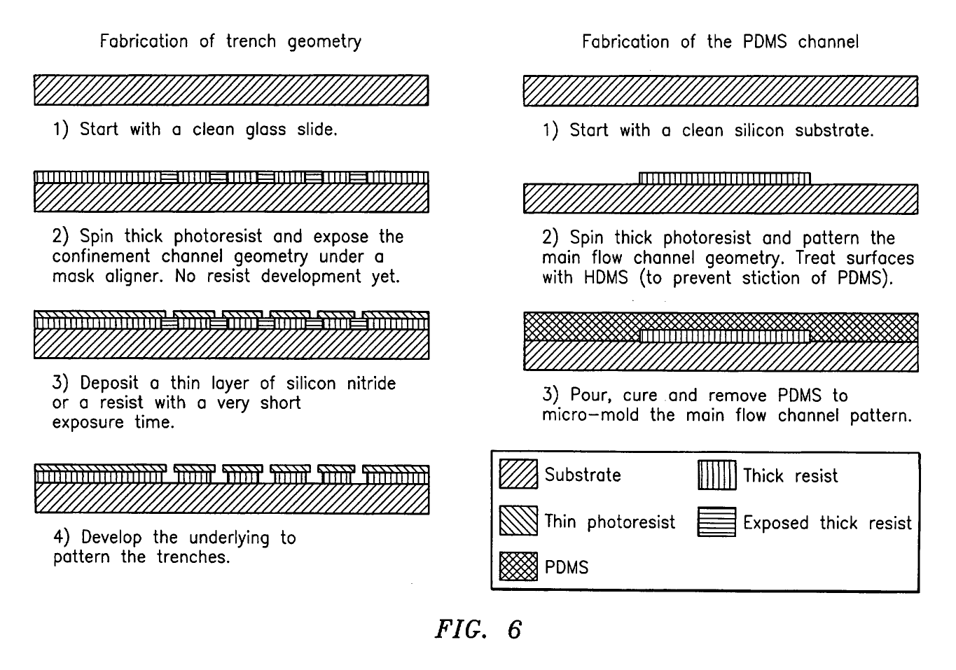

Fabrication of the microfluidic devices is based on the already established soft lithography techniques. FIG. 6 illustrates the basic steps of the microfabrication process. Essentially, glass microscope slides can be used as substrates, and photosensitive polymers help define the channel geometries. Any necessary general surface treatments can be performed after the fabrication steps shown in FIG. 6, and prior to bonding PDMS to the confinement trench substrate.

Fabrication of the microfluidic device may typically comprise the steps of:

(1) providing a glass slide;

(2) fabricating a layer comprising at least one microfluidic trench on the glass slide, wherein an opening of the at least one microfluidic trench is opposite the surface of the glass slide;

(3) providing a roof structure layer on the layer comprising the at least one microfluidic trench, wherein said roof structure comprises a plurality of openings; and

(4) providing a top layer comprising a flow channel, wherein said flow channel is open to the roof structure.

In one embodiment, the top layer comprises poly-dimethylsiloxane and the flow channel comprises an inlet and an outlet (as depicted in FIG. 1). The inlet may also comprise means for introducing a controlled concentration gradient of the fluid into the flow channel. A substance may be introduced into the at least one microfluidic trench and trapped within the microfluidic trench by sealing each end of the microfluidic trench. The fluid introduced into the flow channel is introducible into the at least one microfluidic trench through the roof structure layer to contact the substance, typically by diffusion.

Side input ports are used to directly connect to the trench structures and the cells or living moiety may be introduced by a pressure driven system or a syringe pump. As discussed above, one potential challenge the inventor faced was determining the best mechanism to keep a particular cell inside a given trench, as the cell may either swim up into the main flow channel (and get caught in the stream to be swept out of the device) or simply swim out of the trench into the side injection ports.

There are several ways the inventor has developed to prevent the cells from swimming up into the main flow channel, including the use of a “jail bar” structure (as shown in FIG. 3), in which rectangular slot openings narrower than the cell dimensions are integrated within a roof over the at least one microfluidic trench to allow diffusion to take place while physically confining the cells. It is believed that any roof structure that prevents the cells or living moiety from escaping from the at least one microfluidic trench into the main flow channel while allowing diffusion of the chemotactic agent into the at least one microfluidic trench may be usable in the practice of the invention.

As discussed above, preventing the cells from swimming out of the trench on the side may be accomplished by means of integrated microfluidic valves or additional flow streams along the edges of the main flow channel carrying a chemotactic repellent. Intentionally introducing tiny air bubbles on the side boundaries of the trenches may also be used to trap the cells within the microfluidic trench.

With this application of microfluidics technology, multiple trenches can be formed on the same substrate, and multiple cell species can be supported simultaneously. The capability to work with different cell species on the same device opens the door to the creation of “physiological system” models, where a simplified version of a multicellular organism could be simulated. When the cells of interest are bacteria, the effects of chemicals, new drugs or antibacterial agents can be studied simultaneously on different subspecies. Real time observation of chemotaxis behavior on multiple cell types allows these devices to be used as drug discovery platforms.

Chemotaxis itself can also be used as a cellular manipulation scheme to coax the cells to specific locations within the trenches where interactive sensors are located. In this respect, these chemotaxis chambers are ideal candidates for housing the nano-scale sensing and signaling posts that form the basis of this proposal. For instance, if the posts are located at the center of a confinement trench, then a chemotactic attractant can be used to create a concentration profile with a peak at the center of the main flow channel. In this manner, the cells would be drawn to the center of the trench and eventually some would make contact with the posts.

The inventor recognizes that chemotaxis is not the only means to manipulate cells, though it is probably the most “natural” means to do so. After all, motile cells in their natural habitats continuously respond to gradients of chemicals that correspond to specific signals, such as those from other cells, or the presence of food. Nevertheless, the MEMS approach easily accommodates the integration of other cellular manipulation schemes, such as dielectropherosis traps or integrated micro-valves and pumps, into the chemotaxis chambers. When used in conjunction with a suitable chemotaxis profile, such methods will likely prove very effective in localizing individual cells in the vicinity of the integrated nano-posts.

Another appealing aspect of the overall fluidic system of the instant invention is its capability to change on the fly the chemical environment that the cells are subjected to. Hence, once a given cell or bacterium connects with a nano-post structure, an amazing wealth of biologically relevant studies can be conducted.

These microfluidic chambers and associated support hardware will prove invaluable in determining an accurate model for the exact mechanism of chemotaxis in virtually all cell types and species. They would also provide a foundation for understanding complex hormonal and chemical signaling pathways. It is expected that these devices will generate a wealth of new information and knowledge in medical areas ranging from pregnancy to sexual dysfunction, from aging to depression.

One of the preferred uses of these devices is in drug discovery and pharmacological studies. By studying competing chemotactic agents simultaneously on multiple cell species on the same device (where multiple trenches will be filled with different cell types), drug impact studies can be conducted and the potency of new drug candidates can be tested directly on a cellular level. Potential applications include drug testing for immunological disorders, HIV, Anthrax, various forms of cancer, and in stem cell research. The microfluidic device of the invention allows researchers to conduct side-effect testing of drugs simultaneously with potency measurements.

The idea of the trenches can also be generalized to create a network of such geometries integrated within microfluidic channels. Such configurations would allow the creation of complex “societies of cells”, even miniature organs, within these devices. The many uses of such devices would be, for example, in the biomedical engineering research and health industry.

These microfabricated devices may also be used in conjunction with neurotrophic growth factors in order to create controlled neural networks on substrates. Hippocampal neurons from rats and mice could be placed in the aforementioned trenches and their axon growth could be directed using specific concentration gradient patterns of the growth factors. Such networks could allow scientists to study the effects of various drugs and/or stimulants on the efficacy of synaptic transmissions. Studies on such debilitating conditions as Alzheimer's and Parkinson's disease can be conducted in much more controlled and easy to observe conditions.

What is claimed is:

1. A microfluidic device for measuring natural motile response of a living moiety to a chemotactic agent, comprising:
a) a flow channel for transporting the chemotactic agent through the microfluidic device;
b) at least one microfluidic trench arranged beneath and substantially perpendicular to the flow channel, wherein the living moiety is introducible into the at least one microfluidic trench; and
c) means for measuring the response of the living moiety to the chemotactic agent;
wherein the chemotactic agent is introducible into the at least one microfluidic trench to expose the living moiety to the chemotactic agent, and wherein the flow of the chemotactic agent over the at least one microfluidic trench creates a hydrodynamic stagnation of flow within the at least one microfluidic trench.

2. The microfluidic device according to claim 1, wherein the chemotactic agent is introducible into the at least one microfluidic trench by diffusion.

3. The microfluidic device according to claim 1, wherein the at least one microfluidic trench has a length dimension that is much larger than its dimensions for width and depth.

4. The microfluidic device according to claim 1, wherein the living moiety is selected from the group consisting of bacteria and cell species.

5. The microfluidic device according to claim 1, wherein the at least one microfluidic trench allows for full motility of the living moiety within the at least one microfluidic trench.

6. The microfluidic device according to claim 1, comprising means for trapping the living moiety in the at least one microfluidic trench.

7. The microfluidic device according to claim 6, wherein the means for trapping the living moiety in the at least one microfluidic trench comprises sealing each end of the microfluidic trench.

8. The microfluidic device according to claim 7, wherein each end of the microfluidic device is sealed by means of a mechanical microvalve or an air valve.

9. The microfluidic device according to claim 1, wherein the means for trapping the living moiety in the at least one microfluidic trench comprises a roof structure over the at least one microfluidic trench.

10. The microfluidic device according to claim 9, wherein the roof structure comprises a patterned substrate surface that traps the living moiety within the at least one microfluidic trench while allowing the chemotactic agent to be introduced into the at least one microfluidic trench.

11. The microfluidic device according to claim 10, wherein the roof structure comprises a semi-permeable membrane.

12. The microfluidic device according to claim 11, wherein the semi-permeable membrane is selected from the group consisting of polycarbonate and polyethylene.

13. The microfluidic device according to claim 1, wherein the microfluidic device communicates with at least one additional microfluidic device as part of a system of devices through the control of microvalves and micropumps.

14. The microfluidic device according to claim 1, comprising temporally varying electric fields, wherein the temporally varying electric field are applied to group, separate or select specific living moieties within the at least one microfluidic trench.

15. The microfluidic device according to claim 1, further comprising a means for creating a controlled concentration gradient of the chemotactic agent prior to introducing the chemotactic agent into the flow channel.

16. A method of using a microfluidic device to measure natural motile response of a living moiety to a chemotactic agent, the method comprising the steps of:
a) transporting the chemotactic agent through the microfluidic device in a flow channel;
b) introducing at least one living moiety into at least one microfluidic trench arranged beneath and substantially perpendicular to the flow channel; and
c) measuring the response of the at least one living moiety to the chemotactic agent;
wherein the chemotactic agent is introduced into the at least one microfluidic trench to expose the at least one living moiety to the chemotactic agent, and wherein the flow of the chemotactic agent over the at least one microfluidic trench creates a hydrodynamic stagnation of flow within the at least one microfluidic channel.

17. The method according to claim 16, wherein the chemotactic agent is introduced into the at least one microfluidic trench by diffusion.

18. The method according to claim 16, wherein the at least one living moiety is selected from the group consisting of bacterium and cell species.

19. The method according to claim 16, wherein the at least one microfluidic trench allows for full motility of the at least one living moiety within the at least one microfluidic trench.

20. The method according to claim 16, wherein the at least one living moiety is trapped in the at least one microfluidic trench.

21. The method according to claim 20, wherein the at least one living moiety is trapped in the at least one microfluidic trench by sealing each end of the microfluidic trench.

22. The method according to claim 21, wherein each end of the microfluidic trench is sealed by means of a mechanical microvalve or an air valve.

23. The method according to claim 16, wherein the at least one living moiety is trapped in the at least one microfluidic trench by positioning a roof structure over the at least one microfluidic trench.

24. The method according to claim 23, wherein the roof structure comprises a patterned substrate surface that traps the at least one living moiety within the at least one microfluidic trench while allowing the chemotactic agent to be introduced into the at least one microfluidic trench.

25. The method according to claim 24, wherein the roof structure comprises a semi-permeable membrane.

26. The method according to claim 25, wherein the semi-permeable membrane is selected from the group consisting of polycarbonate and polyethylene.

27. The method according to claim 16, wherein a controlled concentration gradient of the chemotactic agent is introduced into the flow channel.

28. The method according to claim 16, wherein the at least one living moiety is a bacteria and the microfluidic device is used to study bacterial chemotaxis.

29. The method according to claim 16, wherein the microfluidic device is used to study cancer metastatis.

30. The method according to claim 16, where the microfluidic device is used to study stem cell populations.

31. The method according to claim 30, wherein the microfluidic device is used to study neuronal stem cells and their ability to communicate with one another in a controlled environment.

32. The method according to claim 16, wherein the microfluidic device is used to study immune response.

33. A method of fabricating a microfluidic device comprising the steps of:
a) providing a glass slide;
b) fabricating a layer comprising at least one microfluidic trench on the glass slide, wherein an opening of the at least one microfluidic trench is opposite the surface of the glass slide;
c) providing a roof structure layer on the layer comprising the at least one microfluidic trench, wherein said roof structure comprises a plurality of openings; and
d) providing a top layer comprising a flow channel, wherein said flow channel is open to the roof structure;
wherein a fluid introduced into the flow channel is introducible into the at least one microfluidic trench through the roof structure layer.

34. The method according to claim 33, wherein the top layer comprises poly-dimethylsiloxane and the flow channel comprises an inlet and an outlet.

35. The method according to claim 34, wherein the inlet comprises means for introducing a controlled concentration gradient of the fluid into the flow channel.

36. The method according to claim 35, comprising a means for introducing a substance into the at least one microfluidic trench.

37. The method according to claim 36, further comprising sealing each end of the microfluidic trench to trap the substance within the microfluidic trench.

38. The method according to claim 33, wherein the fluid is introduced into the at least one microfluidic trench by diffusion.
